(19) United States
(12) Patent Application Publication
(10) Pub. No.: US 2024/0219345 A1
(43) Pub. Date: Jul. 4, 2024

(54) SAMPLE TRANSFER METHOD AND SYSTEM

(71) Applicant: UNIVERSITY OF TASMANIA, Sandy Bay, Tasmania (AU)

(72) Inventors: Vipul GUPTA, Sandy Bay, Tasmania (AU); Brett PAULL, Sandy Bay, Tasmania (AU); Arushi MANCHANDA, Sandy Bay, Tasmania (AU)

(21) Appl. No.: 18/557,893

(22) PCT Filed: Apr. 29, 2022
(86) PCT No.: PCT/AU2022/050398

§ 371 Date: Oct. 27, 2023

(30) Foreign Application Priority Data
Apr. 30, 2021 (AU) 2021901294

(51) Int. Cl. G01N 27/447; G01N 1/40; G01N 33/487

(52) U.S. Cl. CPC G01N 27/44743; G01N 1/40; G01N 27/44791; G01N 33/48707; G01N 2001/4038

(57) ABSTRACT

Methods and systems for the transfer of samples collected on swabs (or similar sample collectors) onto particular types of substrates for subsequent analysis or other processing. The method involves contacting the sample collector containing a sample including the charged substance with the electrophoresis matrix; applying an electric field across the electrophoresis matrix to effect transfer of the charged substance from the sample collector to the electrophoresis matrix. The useful systems may assist in the performance of an operation on a charged substances within an electrophoresis system. Such systems may include a first electrolyte reservoir including a first electrode; a second electrolyte reservoir including a second electrode; a thread extending between the first and second electrolyte reservoirs; a third reservoir through which the thread passes, and a controller for controlling the application of an electric field across the thread so as to effect movement of a charged substance along the thread.

### TECHNICAL FIELD

The present application relates to methods and systems for the transfer of samples collected on swabs (or similar sample collectors) onto particular types of substrates for subsequent analysis or other processing. Such transfers are particularly relevant to collection and concentration of charged components in a sample by electrophoresis. The present application also relates to useful systems, components and processes that may assist in performing an operation on a charged substance taken from a sample. Such systems, components and methods are relevant to the high-throughput analysis of swabbed samples, such as in the detection of chemical residues, biochemicals, and biomolecules, including cells and viruses, and in other broader applications.

### BACKGROUND OF THE INVENTION

Swab-based sample collection is one of the most widely used methods for biochemical, pharmaceutical, forensic, environmental, and other analytical procedures because of its low-cost, ease of use, and the ability to collect both liquid and dry samples from a variety of surfaces. Collected swabs are usually transported to a remote laboratory, where the swabbed sample is desorbed into a solvent to allow further testing using different analytical instruments. The transport of the swab from the sample collection site to a laboratory poses significant delays, which hampers timely analysis and decision-making, especially in life-threatening circumstances, such as disease diagnosis, terrorism threats, epidemics, or pandemics, and so forth. Recently, significant advancement has been made in developing various point-of-care (POC) analysis systems for on-site analysis to minimise the transport delays. However, they have been rarely integrated with a swab-based collection system and still rely on the desorption of the swabbed sample into a solvent, which is then injected into these systems. This procedure not only increases the risk of sample contamination by introducing additional manual steps, it also dilutes the collected sample hampering its qualitative and quantitative analysis (especially considering the already lower sensitivity of the most POC systems).

Traditionally, POC analytical devices have been developed using microchannels as fluidic conduits. More recently, other substrates such as paper and textiles have been considered for providing fluidic conduits. Whilst those alternatives have been studied to some extent, microfluidic textile analytical devices (μTADs) have not yet been developed into complete systems that allow for sample collection through to sample analysis, including integrated with suitable sample collection devices, such as swabs.

Hence, an object of some embodiments of the present application is to develop new techniques to assist with the on-site direct analysis of swabbed samples.

It has also been recognised that it would be advantageous for the system to be able to perform multiple parallel runs either on a single swab or from multiple swabs, to achieve multiplexed or high throughput analysis. Providing such functionality would minimise the average sample analysis time in situations where large numbers of samples are being taken for laboratory analysis, such as epidemics and pandemics. Microfluidic systems used historically for sample analysis, including microfluidic textile analytical devices, have been restricted to the use of electrode coupled initial and terminating reservoirs. This configuration has limited their use by limiting the number of activities that can be performed during a single run and poses the challenges of electrode fouling due to their direct contact with the corrosive samples and reagents that have to be introduced in the same reservoirs as the electrodes.

It is an object of some embodiments described herein to provide a new system and techniques that facilitate multi-step analysis and/or minimises the risks of electrode fouling. It would also be advantageous for preferred embodiments to enable the performance of complex analytical procedures, which are often required in real-world settings. Moreover, since some analytical devices rely on the application of high voltages, it would further be advantageous for embodiments to be able to be operated more safely and minimise the risk of user exposure to live electrodes.

Owing to the widespread use of swabs and microfluidic devices in disease diagnosis, forensic investigations, threat analysis, pharmaceutical analysis, and so forth, the developed technology is of utility in various applications.

### SUMMARY OF THE INVENTION

According to a first aspect, the present application provides a method for the transfer of a charged substance from a sample on a sample collector to an electrophoresis matrix, the method comprising:
contacting the sample collector containing a sample including the charged substance with the electrophoresis matrix in the presence of an electrolyte;
applying an electric field across the electrophoresis matrix to effect transfer of the charged substance from the sample collector to the electrophoresis matrix.

The above method effects the transfer of the charged substance directly from the sample collector to the electrophoresis matrix, without an intervening transfer or diffusion into a bulk electrolyte and out of a bulk electrolyte. This allows for the efficient and quick transfer of charged substance from the sample collector to the electrophoresis matrix. This can also be achieved with a concentration of the charged substance. Concentration is achieved without a selective membrane positioned between the sample collector and the electrophoresis matrix. The sample collector is contacted directly with the electrophoresis matrix.

The electrophoresis matrix is in contact with electrolyte during the application of the electric field. For example, the electrolyte may comprise a volume of the electrolyte—i.e. the “bulk electrolyte” that wets the electrolyte matrix, or the electrolyte may wet the electrolyte matrix by coating or wicking of the electrolyte matrix by the electrolyte. The applicant has surprisingly found that, where the electrophoresis matrix is in a bulk electrolyte, rather than diffusing into the bulk electrolyte, the charged substance follows the pathway of the electric field and transfers from the sample collector directly to the electrophoresis matrix. Wetting of the electrophoresis matrix by the electrolyte in the absence of a volume of bulk electrolyte similarly enables the transfer of a high percentage of the charged substance from the sample collector onto the electrophoresis matrix. Thereafter, the charged substance can be further processed or moved along the electrophoresis matrix as desired. Examples of options for further processing or transfer of the charged substance along the electrophoresis matrix are described herein.

In typical embodiments, the electrophoretic matrix comprises a thread. The sample collector may be in the form of a swab.

According to a second aspect, the present application further provides a system for the transfer of a charged substance from a sample on a sample collector to an electrophoresis matrix, the system comprising components including:
a sample transfer reservoir;
an electrophoresis matrix comprising a thread positioned through the sample transfer reservoir;
a pair of electrodes positioned to enable the application of an electric field across the thread;
wherein the components are positioned such that the sample collector contacts the thread during the application of an electric field across the thread to enable transfer of the charged substance from the sample collector to the thread.

The system may further comprise a receiver for receiving the sample collector (e.g. the swab). In some embodiments the sample transfer reservoir may serve as the receiver for receiving the sample collector, or the receiver may be in the form of a separate feature of the device into which the swab is positioned, before it is moved into contact with the thread in the sample transfer reservoir.

It is also noted that the system or device may be in the form of a cartridge. Alternatively, the system may include a cartridge that provides one or more of the components of the system described above. Further details of this cartridge-type arrangement and other possible arrangements are described below.

The use of an electrophoretic matrix that creates a pathway for an electric field in an open system, such as a thread in particular, offers various advantages over conventionally used microchannels. These include low-cost, high flexibility, high mechanical strength even under wet conditions, reusability, disposability, and ease of functionalisation and arrangement into complex 2D and 3D structures. Moreover, thread-based devices and their equivalents do not require pumping systems, and allow easy manipulation and on-line modification of the sample. The on-line modification is due in part to the “open” nature of the thread. Specifically, the environment may be modified along the thread without restriction (e.g. another substance can be added, a sample taken etc)—this contrasts to a “closed” capillary which is not open to the environment and cannot be modified in the same manner (e.g. another substance cannot be added into the channel without an access opening in the capillary). Whilst this is a clear advantage of thread-based systems or their equivalents, such microfluidic textile analytical devices have not, until now, been successfully integrated with swab-based collection devices. The simple transfer of a swabbed sample to a thread or equivalent electrophoretic matrix as described in the present application, with a high degree of sample transfer and minimal loss, circumvents the need for swab transport and/or sample desorption into a solution, providing a low concentration sample solution.

The method of the present application in some embodiments involves a simple step of placing the swab in contact with the thread either through a designated sample transfer reservoir or sample receiver of the analytical device, where a quantitative transfer, or near-quantitative transfer, of the charged substances (including potential analytes) is performed from the swab onto the thread. The transfer is achieved by simply bringing the swab and the thread into direct contact and applying a voltage potential across the thread. The test work presented herein indicates that close to 100% recovery of analytes can be achieved. These results have been achieved with a range of different types of analytes, a range of swabs (both dry and wetted by electrolyte), a range of different thread materials, and in the presence of a range of sample matrices. The degree of transfer may be at least 50%, 60%, 70%, 80% or at least 90% of the target charged substances from the sample collector to the electrophoresis matrix. The transfer of the charged substance from the sample collector to the electrophoresis matrix in some embodiments can occur to the substantial exclusion of uncharged substances in the sample. This is achieved by suppressing electro-osmotic flow, through which charged substances can be transferred to the electrophoresis matrix and not the uncharged substances. The transferred analytes have been further successfully manipulated on the threads using procedures, such as isotachophoresis, electrophoresis, sample splitting, or physical movement of the thread itself. In other embodiments, where it is desired to transfer uncharged substances to the electrophoresis matrix in addition to charged analytes, it may be possible to modify the conditions to achieve this.

In addition to providing an effective means for transferring charged substances from a sample to the thread or equivalent electrophoretic matrix, further developments have been made in the overall device arrangement that provides additional functionality and convenience for microfluidic textile (thread-based) analytical devices.

According to a third aspect, the present application provides an electrophoresis system comprising:
a first electrolyte reservoir comprising a first electrode;
a second electrolyte reservoir comprising a second electrode;
a thread extending between the first and second electrolyte reservoirs;
a third reservoir through which the thread passes; and
a controller for controlling the application of an electric field across the thread so as to effect movement of a charged substance along the thread;
wherein the third reservoir enables either (a) the charged substance to be loaded onto the thread in the third reservoir, or (b) an operation to be performed on the charged substance transferred to the third reservoir following movement of the charged substance along the thread on the application of the electric field.

In some embodiments, the electrophoresis system of the third aspect comprising:
a first electrolyte reservoir comprising a first electrode;
a second electrolyte reservoir comprising a second electrode;
a thread extending between the first and second electrolyte reservoirs;
a third reservoir through which the thread passes between the first and second electrolyte reservoirs; and
a controller for controlling the application of an electric field across the thread so as to effect movement of a charged substance along the thread in a direction towards the second electrolyte reservoir;
wherein the third reservoir enables either (a) the charged substance to be loaded onto the thread in the third reservoir, or (b) an operation to be performed on the charged substance transferred to the third reservoir following movement of the charged substance along the thread on the application of the electric field.

The applicant has devised a new electrophoretic system arrangement comprising at least three reservoirs—including separate reservoirs for the first and second electrodes and at least one other reservoir, which may be an intermediate reservoir (the third reservoir) positioned along the thread between the first and second electrode-containing reservoirs. The third reservoir is free of any electrode. There may be one or more additional reservoirs in addition to the third reservoir. The additional reservoirs may be positioned along the thread between the first and second electrode-containing reservoirs. In alternative embodiments, the additional reservoirs (or some of these reservoirs) may be positioned before or after the first and second electrode-containing reservoirs. The application of an electric field between the first and second electrodes results in the application of an unbroken electric field across the thread extending through the third reservoir. If the third reservoir is positioned before or after the first and second reservoirs, the electrophoretic force would need to be strong enough to pull the charged substances from that reservoir towards the first (and second) reservoirs.

In cases where system contains only three reservoirs, the sample may be loaded onto the thread in the first reservoir, and then the charged substance can be transported under the influence of the electric field along the thread towards and into the third reservoir. The charged substance may then be desorbed from the thread and into the bulk electrolyte in the third reservoir, where an operation may be performed. “Operation” refers to a chemical analysis, detection, coupling or modification of the charged substance. Examples include analyte detection, analyte modification, coupling of the charged substance to a marker, a chemical reaction or a transformation involving the charged substance, complex detection involving the charged substance (e.g. PCR) and so forth. This system provides flexibility in terms of the functionality of the system and the ability to perform operations in a liquid state, within a bulk electrolyte, rather than in the solid state or otherwise.

In an alternative arrangement for the three-reservoir system, the sample may be loaded onto the thread in the third reservoir, and then passed along the thread through electrophoresis. The charged substance that is moved along the thread between the third reservoir and the second electrolyte reservoir through the application of the electric field may then be used in any suitable process or subjected to any desired process. As one example, a zone of the thread following the application of the electric field may be cut away, and the cut portion subjected to further processing to recover the charged substance. Alternatively, an operation can be performed on the charged substance either on the thread or once it has been desorbed from the thread, either within a reservoir of the system, or otherwise.

In alternative embodiments, the system may comprise two or more reservoirs between the first and second reservoirs—yielding a system with four or more reservoirs (per thread). One of the two reservoirs positioned between the first and second reservoirs may be a sample loading reservoir for loading sample onto the thread. Sample loading may be achieved using the process described above for the first and second aspects, or otherwise. The second of the reservoirs may be an operation reservoir, which is positioned along the thread between the sample loading reservoir and the second electrolyte reservoir. In the operation reservoir an operation can be performed involving the charged substance in the operation reservoir.

In use, the reservoirs may contain electrolyte, and the thread is wetted with electrolyte or is coated in a conductive substance such as a hydrogel to provide an electrical pathway along the thread between the reservoirs. Charged substances may be desorbed into the electrolyte in particular reservoirs, as required by the process being undertaken. The system described herein allows for multiple operations or processes to be performed in multiple reservoirs, using a thread-and-reservoir arrangement, and the application of an electric field to transfer charged substance(s) between reservoirs. The charged substances can be desorbed from the thread into the bulk electrolyte, and re-loaded onto the thread as required. In the past, capillaries have been considered for moving substances from one bulk electrolyte to another. However, the present system provides flexibility in terms of providing the option to either retain the charged substance on the thread (concentrated), or to desorb into a bulk solution.

The third reservoir (and each additional reservoir) is free of any electrode, while still maintaining the electric circuit between the electrode carrying reservoirs. Where there are dedicated sample loading reservoirs and operation reservoirs, each of these reservoirs is free of any electrode.

The system, or components of the system, may be provided in a cartridge format. Accordingly, there may be provided a cartridge for use in an electrophoresis instrument, the cartridge comprising:
a first electrolyte reservoir comprising a first electrode;
a second electrolyte reservoir comprising a second electrode;
a thread extending between the first and second electrolyte reservoirs; and
a third reservoir through which the thread passes between the first and second electrolyte reservoirs, within which either (a) a charged substance may be loaded onto the thread, or (b) an operation can be performed on charged substance following transfer of the charged substance along the thread to the third reservoir.

The cartridge may further comprise electrolyte for each of the first electrolyte reservoir and the second electrolyte reservoir. The cartridge may additionally comprise electrically conductive reagent for the third reservoir.

Further features of the cartridge correspond to the exemplified features described above for the third aspect of the invention. Accordingly, the cartridge may comprise one or more additional reservoirs. The third reservoir may be a sample loading reservoir within which a charged substance may be loaded onto the thread in use, and an additional reservoir may function as an operation reservoir within which an operation can be performed on the charged substance following transfer from the sample loading reservoir to the operation reservoir. The reagents may be pre-filled within the reservoir. In an alternative arrangement, the reagents may be provided separately, and may be added at the appropriate time to each reservoir. For example, the reagents may be ready for transfer into the respective reservoirs from one or more sealed pods. The contents of the pod(s) may be transferred into the respective reservoir(s) through any suitable means, such as through puncturing the pod(s) at the appropriate time to release the contents of the pod(s) into the reservoir(s).

In some embodiments, the system includes an array comprising multiple sets of said first and second electrolyte reservoirs, first and second electrodes, thread, and third reservoirs (and optionally any further reservoirs). If provided in cartridge form, each cartridge may be for a single set, or a single cartridge may contain multiple sets of the reservoirs, electrodes and thread. In an alternative arrangement, the cartridge may comprise first and second electrolyte reservoirs, with multiple threads spanning between the first and second electrolyte reservoirs, each thread including one or more intermediate reservoirs along its length. This may be referred to as a “thread splitting” arrangement. In this case, each thread shares the first and second electrolyte reservoirs with other threads, but each has its own sample loading reservoir(s) and/or sample operation reservoir(s) or zones.

An array of such components allows for multiple parallel runs to be performed to achieve multiplexed or high throughput analysis. These may be performed on either a single sample (or single sample collector/swab) or from multiple samples (sample collectors/swabs). Multiplexed analysis has also been performed by splitting a single thread into multiple pathways, where each pathway was used to determine a specific marker to provide a more holistic sample analysis and minimise the false positive and negative results that are often obtained when a single marker is analysed. High throughput analysis has been performed by recruiting multiple threads, substantially in parallel, in which each thread was used to perform analysis on an individual swab, minimising the average sample analysis time in situations such as epidemics and pandemics.

As indicated further above, microfluidic textile analytical devices have been restricted to the use of electrode-coupled initial and terminating reservoirs. However, the above-described configuration of the third aspect of the present application has been developed that allows the use of electrode-free reservoirs, facilitating multi-step analysis and minimising the risks of electrode fouling. In key embodiments of the third aspect, there may be one or more additional reservoirs arranged between the initial and terminating electrodes (the first and second electrodes) such that they do not break the electro-fluidic circuit while also allowing independent activities, such as sample introduction, concentration, modification, detection, selective uptake or release, etc. The developed system can facilitate the use of microfluidic textile analytical devices in performing complex analytical procedures, which are often required in real-world settings. Moreover, since microfluidic textile analytical devices use high voltages, the availability of electrode-free reservoirs for sample manipulation would also promote the generation of safer microfluidic textile analytical devices by preventing user exposure to the live electrodes. The present system, while suitably making use of a high voltage potential, uses low current and is designed for safe operation. The voltage potential applied in some embodiments is at least 900 V The current in some embodiments is less than 300 μA.

According to a fourth aspect, there is provided a method for performing an operation on a charged substance, the method comprising:
providing a first electrolyte reservoir comprising a first electrode, a second electrolyte reservoir comprising a second electrode and a thread that extends between the first and second electrolyte reservoirs;
loading a charged substance onto the thread;
moving the charged substance along the thread to an operation reservoir positioned downstream in the flow direction of the charged substance, by applying an electric field across the thread between the first and second electrodes; and
performing an operation involving the charged substance in the operation reservoir.

In preferred embodiments the charged substance is taken from a sample, and is loaded onto the thread in a sample loading reservoir.

There may be more than one operation reservoir (or operation zones) traversed by the thread, and allowing for different operations to be performed in each of said reservoirs (or in each of said zones). The movement of the charged substance along the thread involves the application of an electric field across the thread.

### BRIEF DESCRIPTION OF THE DRAWING(S)

FIG. 1 is a schematic view of the thread-based device in accordance with one embodiment of an invention described herein.

FIG. 2 shows images of swabs and threads following a transfer, or attempted transfer, of a charged substance (fluorescein) from a swab (polyurethane) to a thread (nylon), in accordance with one embodiment of an invention described herein. FIG. 2(a) shows the swab (left side) and thread (right side) following an attempted transfer without direct contact between the swab and the thread. FIG. 2(b) shows the swab (left side) and thread (right side) following an attempted transfer without any applied electric field while the swab and the thread were in direct contact. FIG. 2(c) shows the swab (left side) and thread (right side) following a transfer completed with direct contact between the swab and the thread.

FIG. 3 is a schematic illustration of a thread-based device incorporating a cartridge design in accordance with one embodiment of an invention described herein.

FIG. 4 is a schematic illustration of a thread-based device incorporating a multiplexed, cartridge design in accordance with another embodiment of an invention described herein.

FIG. 5 shows images demonstrating the transfer of a range of charged substances from a sample collector (swab) to a thread using electrophoresis in accordance of some embodiments of an invention described herein. In FIG. 5(a), the left-hand image shows a fluorescent image of the swab after the transfer is effected by electrophoresis, and the right-hand image (thread line) shows the positioning of the fluorescent fluorescein on the thread post-transfer and post-electrophoretic focusing. FIG. 5(b) shows the swab for the alkaloid composition post-transfer (left-half image) and the right-half image shows the alkaloids on the thread post-transfer and post-focusing. FIG. 5(c) shows the swab for the labelled protein post-transfer on the left, and the thread post-transfer on the right showing the presence of the protein.

FIG. 6 shows images demonstrating the transfer of a charged substance, fluorescein, to threads of different materials including (a) nylon, (b) mercerised cotton, (c) cotton and (d) polyester, in accordance with some embodiments of an invention described herein. In each sub-figure, a fluorescent image of the swab after the transfer is shown on the left and a fluorescent image of the transferred and focused analyte band on the thread is shown on the right.

FIG. 7 shows images demonstrating the transfer of a charged substance (in particular, fluorescein) from different swab materials, (a) polyurethane and (b) cotton, onto a thread using electrophoresis, in accordance with some embodiments of an invention described herein. In each sub-figure, a fluorescent image of the swab after the transfer is shown on the left and a fluorescent image of the transferred and focused analyte band on the thread is shown on the right.

FIG. 8 is a schematic illustration of a reservoir design for the sample loading reservoir of one embodiment of the invention.

FIG. 9 shows images of the swab and thread junctions before (shown on left) and after (shown on right) the transfer of a charged substance (fluoresceine) from a swab (polyurethane) to a thread (nylon) in the absence of any additional liquid in the sample transfer reservoir, in accordance with one embodiment of the invention described herein.

FIG. 10 shows images of the swab and thread junctions before (shown on left) and after (shown on right) the transfer of a charged substance (fluoresceine) from a swab (polyurethane) to a thread (nylon) in the absence of any additional liquid in the sample transfer reservoir, in accordance with one embodiment of the invention described herein. FIG. 10(a) shows images of the swab and thread junction for the transfer of a liquid sample from a pre-wetted swab. FIG. 10(b) shows images of the swab and thread junction for the transfer of a liquid sample from a dry swab. FIG. 10(c) shows images of the swab and thread junction for the transfer of a powder sample from a dry swab.

FIG. 11 shows images of the swab and thread junctions before (shown on left) and after (shown on right) the transfer of a charged substance (fluoresceine) from a swab (polyurethane) to a thread (nylon), in accordance with one embodiment of the invention described herein. FIG. 11(a) shows images of the swab and thread junction for the transfer of a liquid sample dried on a plastic surface and swabbed with a pre-wetted swab. FIG. 11(b) shows images of the swab and thread junction for the transfer of a liquid sample dried on a plastic surface and swabbed with a dry swab. FIG. 11(c) shows images of the swab and thread junction for the transfer of a liquid sample dried on a metallic surface and swabbed with a pre-wetted swab. FIG. 11(d) shows images of the swab and thread junction for the transfer of a liquid sample dried on a metallic surface and swabbed with a dry swab. FIG. 11(e) shows images of the swab and thread junction for the transfer of a liquid sample dried on a wooden surface and swabbed with a pre-wetted swab.

FIG. 12 shows images of the swab and thread junction before (shown on left) and after (shown on right) the transfer of a charged substance (fluoresceine) from a swab (polyurethane) to a thread (nylon) from spiked saliva samples in the presence of a cell lysis buffer, in accordance with one embodiment of the invention described herein.

FIG. 13 shows images of the transfer, separation, and concentration of a charged substance (fluorescently tagged DNA) present in a complex sample (defibrillated sheep blood) from a swab (polyurethane) to a thread (nylon), in accordance with one embodiment of the invention described herein. FIG. 13(a) shows the image of the swabbed spiked blood sample. FIG. 13(b) shows the image of the separated blood cells and haemoglobin on the thread. FIG. 13(c) shows the microscopic image of the lysed cells on the thread. FIG. 13(d) shows the image of the focused fluorescently tagged DNA band on the thread.

FIG. 14 shows images for splitting a charged substance (fluorescein) transferred from a swab (polyurethane) onto two threads (nylon) each with a discrete operational reservoir, in accordance with one embodiment of the invention described herein. FIG. 14(a) shows the arrangement of the splitting threads with their individual operational reservoirs. FIG. 14(b) shows the image of the swab in direct contact with a single thread. FIG. 14(c) shows the image of the fluorescent analyte split onto two threads.

### DETAILED DESCRIPTION OF PREFERRED EMBODIMENTS

As described above, the present application provides a method for the transfer of a charged substance from a sample on a sample collector to an electrophoresis matrix, the method comprising:
contacting the sample collector containing a sample including the charged substance directly with the electrophoresis matrix;
applying an electric field across the electrophoresis matrix to effect transfer of the charged substance from the sample collector to the electrophoresis matrix.

In preferred embodiments, the electrophoresis matrix is a thread. More generally, the electrophoresis matrix may be a matrix that provides a directional pathway for electro-osmotic flow from one location to another. Examples of suitable matrices are outlined below.

The term “thread”, which may alternatively be referred to as a fibre, refers to any natural or synthetic fibre. The thread may be in the form of one or more threads aligned parallel to one another, or coiled, wound, braided or intertwined. In some embodiments the thread is a single thread. Examples of suitable materials for forming the thread include natural materials such as cotton and silk, and synthetic materials including polymers such as nylon or polyurethane. The thread may generally be of any length and diameter, and may be hollow or solid. While any length or diameter may be used, the diameter may typically be not more than 10 mm in diameter, preferably not more than 9, 8, 7, 6, 5, 4, 3, 2 or 1 mm in diameter. If the thread comprises coiled, wound, braided or intertwined multi-thread components, the diameter refers to the total thread diameter. In another arrangement contemplated herein, the thread may be in the form of a multi-thread network, such as a net, or may include multi-thread network section(s) and single-thread section(s). The thread may be untreated or may be chemically treated to adjust the hydrophilicity/hydrophobicity, to be more or less hydrophilic or hydrophobic. The thread may be functionalised or unfunctionalized. The thread may be coated or uncoated.

In some embodiments, the thread is free of a separate channel-forming substrate. By way of explanation, some techniques in the prior art may rely on channels formed in a block or substrate, into which a thread may be positioned. However, in the present application, the thread itself forms a pathway for an electrolyte solution and for the passage of charged substances under the application of an electric field.

In preferred embodiments, the sample collector is in the form of a swab. A swab is a pad, piece or sheet of material that is able to be wiped or touched to a surface to effect the transfer of a sample (substances) from the surface to the swab to facilitate collection of the sample. The “material” of the swab may be in the form of a fabric (woven, non-woven, felted or otherwise), in the form of a soft block (e.g. a foamed resilient pad or block of any suitable shape), paper, or otherwise. The swab may be formed from natural or synthetic material. Notable examples are polyurethane swabs, flocked nylon swabs, and cotton swabs. Synthetic polymer swabs may be preferred, such as polyurethane swabs.

In preferred embodiments, the method comprises transferring the sample from the sample collector to the electrophoresis matrix or thread without an intervening transfer into a solution. The transfer is direct and autonomous from human interaction.

In preferred embodiments, at least 50% of the charged substance is transferred from the sample collector to the electrophoresis matrix. The amount may be at least 80% of the charged substance/analyte or at least 90% of the charged substance/analyte. The test results shown herein demonstrate that one can transfer substantially all of the charged substance/analyte from the sample collector to the electrophoresis matrix. Expressed another way, the method is able to effect quantitative transfer of the charged substance/analyte from the sample collector to the electrophoresis matrix.

The method involves the application of the electric field in the presence of an electrolyte. The presence of an electrolyte refers to contact of the electrolyte with the electrophoresis matrix. This provides the required electrical pathway for the application of an electric field and the transfer of the charged substance to the electrophoresis matrix under the influence of the electric field. Any electrolytes known in the art may be used. Electrolytes may comprise ionic substances. The electrolyte may be a liquid electrolyte or a gel electrolyte or otherwise.

The electrolyte may comprise a volume of the electrolyte—i.e. a “bulk electrolyte” that wets the electrolyte matrix, or the electrolyte may be present in a smaller volume that just wets the electrolyte matrix by coating or wicking of the electrolyte matrix by the electrolyte. By a “volume” is meant an amount that exceeds the amount required for wetting the electrophoresis matrix. The actual volume of bulk electrolyte will typically depend on the volume of the reservoir containing the bulk electrolyte. The bulk electrolyte in this case may constitute a drop, aliquot or pool of electrolyte in the reservoir. The reservoir may be filled by at least 10%, 20%, 30%, 40%, 50%, 60% or at least 70% of its volume by the electrolyte. The amount of electrolyte may be, for example, at least 0.1 ml, at least 0.5 ml, at least 1 ml, at least 5 ml or at least 10 ml. It has surprisingly found that, where the electrophoresis matrix is in a bulk electrolyte, rather than diffusing into the bulk electrolyte to be spread throughout at low concentration, the charged substance follows the pathway of the electric field and transfers from the sample collector directly to the electrophoresis matrix. Wetting of the electrophoresis matrix by the electrolyte (e.g. by wetting or wicking of the matrix with electrolyte) similarly enables the transfer of a high percentage of the charged substance from the sample collector onto the electrophoresis matrix.

After transfer of the charged substance to the electrophoresis matrix, it may be desired to perform an electrophoretic process on the charged substance. In that situation, there is the option to use the one electrolyte composition throughout the process. In the alternative, a combination of electrolytes may be used, particularly where it is desired to perform an isotachophoretic separation of charged substances/analytes after transfer of the analyte from the sample collector to the electrophoresis matrix. In that case, in some embodiments, the electrolyte used in the sample transfer reservoir is a terminating electrolyte. A terminating electrolyte may alternatively be referred to as a trailing electrolyte. The term is well understood in the art of the invention. The choice of terminating electrolyte may depend on the charged substance or analyte to be subjected to the transfer. Further, if focusing or concentration of the charged substance, or a target substance among the charged substances, is to be performed, then the selection of the terminating electrolyte may be impacted by the identity of the target substance and/or the leading electrolyte. The combination of a terminating electrolyte and a leading electrolyte impacts on the isotachophoretic separation or concentration of the relevant analyte(s).

The above method may involve the application of the electric field in the presence of the electrolyte; wherein a higher proportion of the charged substance transfers to the electrophoresis matrix than is transferred into the electrolyte (e.g. the bulk electrolyte). Typically, the charged substance transfers directly from the sample collector to the electrophoresis matrix or thread, without a separate transfer into the bulk electrolyte and subsequently out of the bulk electrolyte and onto the electrophoresis matrix. The electric field passes through the electrophoresis matrix, and thread in particular, providing an electric field pathway for the transfer of the charged substances from the sample collector directly to the thread, rather than the substance transferring into the bulk electrolyte. The thread, or similarly narrow matrix, provides a focused exit route for the charged substances away from the sample collector.

Any type of sample that contains a charged substance or substances may be used as the sample. Examples include biological samples, pharmaceuticals, environmental samples (e.g. soil) and so forth. One type of sample that may suitably be subjected to the method is a biological sample. Examples of suitable biological samples that may be subjected to the method include saliva, blood, cells, cell lining and mucous.

The term “charged substance” refers to a substance that has a charged state or is polarised in the relevant conditions such that it can move under the influence of an electric field. As an example, the charged substance may be a substance that is ionisable in water. The charged substance may be a charged pharmaceutical, a charged analyte (e.g. a possible contaminating species in an environmental material), or a charged biomolecule, among other examples. In one embodiment, the charged substance may be a charged biomolecule. The charged biomolecule may be selected from polynucleotides, polypeptides, proteins or various combinations thereof. The biomolecules may be natural or synthetic. Polynucleotides may comprise of DNA, RNA or a combination of DNA and RNA. The polynucleotides may be single stranded or double stranded. Double stranded polynucleotides are those in which all the bases are paired with a complementary base on a second polynucleotide strand. For example, some of the single stranded polynucleotides may comprise a sequence complementary to other single stranded polynucleotides. The polynucleotides may also have a combination of single and double stranded portions wherein only a subset of the bases are engaged in complementary base-pairing.

The polynucleotides may comprise of 10 to 1000 nucleotides. For example, the polynucleotides may comprise 10 to 50 nucleotides, 10 to 100 nucleotides, 10 to 250 nucleotides, 10 to 500 nucleotides, 100 to 1000 nucleotides, 250 to 1000 nucleotides, or 500 to 1000 nucleotides.

In some embodiments, the method further comprises:
subjecting the charged substance transferred onto the electrophoresis matrix to electrophoresis to separate a target analyte that forms a component of the charged substance (or constitutes the charged substance) into a focused band on the electrophoresis matrix.

The above method allows for the clean-up of a test analyte from a complex material, such as a complex biological mixture. The clean-up of the test analyte may, where desired, allow for direct analysis to be performed on the electrophoretic matrix as it passes through a detection zone of the electrophoretic matrix (e.g. thread).

In some embodiments, the separated target analyte is concentrated on the electrophoresis matrix, or thread in particular, through the electrophoretic process. The specific form of electrophoresis may be isotachophoresis.

The location of concentration, or the time-period taken for the concentrated target analyte to reach a particular location, enables the target analyte to be separated from other components in the charged substance. This also allows for concentration of a particular charged substance (or target analyte) to be concentrated in one location on the electrophoresis matrix, such as the thread. As the electrophoresis matrix is an “open system”, such as a thread, the thread can be divided at the required location to separate the concentrated region of target analyte. Otherwise, if the location of sample transfer is performed at a sample transfer zone of the thread, then at a spaced location from the sample transfer zone, analysis can be performed on the thread to detect for the target analyte. The zone at which this detection is performed may be described as a detection zone. The detection may be of any suitable type, such as PCR analysis, microanalytical techniques or otherwise. The detection step may involve RNA amplification if required, according to any process known in the art, including reverse-transcription loop-mediated isothermal amplification (RT-LAMP).

The method described above may further comprise the step of:
detecting for the presence of the target analyte.

For methods performed on biological samples, the method may comprise: contacting the sample collector containing the sample with a lysis buffer to lyse the cells present in the biological sample. The lysis buffer suitably also contains electrolyte components for the subsequent electrophoretic separation. If the method involves an isotachophoretic separation, the electrolyte may be a terminating electrolyte.

Further details of the method for performing the charged substance transfer will be described in further detail below with reference to the figures and examples. Additional features of the method will become apparent with reference to the discussion of a system for performing the method. Those system features also provide an indication of preferred features of the method.

Thus, as described above, the second aspect provides a system for the transfer of a charged substance from a sample on a sample collector to an electrophoresis matrix, the system comprising components including:
a receiver for receiving the sample collector;
a sample transfer reservoir;
an electrophoresis matrix comprising a thread positioned through the sample transfer reservoir;
a pair of electrodes positioned to enable the application of an electric field across the thread;
wherein the components are positioned such that the sample collector contacts the thread during the application of an electric field across the thread to enable transfer of the charged substance from the sample collector to the thread.

It is noted that the sample transfer reservoir may serve as the receiver for receiving the sample collector, or the receiver may be in the form of a separate feature of the device into which the swab is positioned, before it is moved into contact with the thread. The receiver may be actuated between one position in which the sample collector is received by the device, and a second position where the sample collector is positioned in contact with the electrophoresis matrix.

It is also noted that the system or device may be in the form of a cartridge. Alternatively, the system may include a cartridge that provides one or more of the components of the system described above. Further details of this cartridge-type arrangement and other possible arrangements are described below.

In some embodiments, the thread has a first end and a second end, and the electrodes are positioned one towards each end of the thread.

In some embodiments, the system comprises an operation reservoir spaced apart from the sample transfer reservoir, and the thread spans the transfer reservoir and the operation reservoir. The thread traverses each of the transfer reservoir and the operation reservoir, and there between. The operation reservoir is a reservoir at which an operation is performed on the charged substance. The operation may be a chemical reaction involving the charged substance, or detection of the charged substance or otherwise. Accordingly, the operation reservoir may be alternatively referred to as a chemical reaction reservoir or a detection reservoir, or otherwise, depending on the operation being performed.

In some embodiments, the system comprises two electrolyte reservoirs. One electrolyte reservoir is positioned to one end of the thread, and the second electrolyte reservoir is positioned to the other end of the thread. Each reservoir is for receiving electrolyte. The electrolytes may be the same. Alternatively, the electrolytes may be different. For an isotachophoretic process, one electrolyte may be a terminating (or trailing) electrolyte and the other may be a leading electrolyte.

The electrodes present as components of the system may be positioned one in each of the two electrolyte reservoirs. The electrodes may be denoted as a positive electrode and a negative electrode, respectively, although the polarity depends on the voltage potential applied across the electrodes.

The reservoirs in each instance are suitably able to hold liquid, such as a liquid electrolyte. In preferred embodiments, the sample loading and operation reservoirs need to allow for bulk liquid electrolyte, or other liquid reagents, to be held, to facilitate charged substance loading and operations to be performed on the charged substance, respectively.

In some embodiments, the system comprises an array of components for performing a plurality of operations (such as detections/analyses/chemical reactions) contemporaneously on one or more samples. The array may comprise at least two sequences, each sequence containing:
at least three reservoirs each,
a thread extending between the reservoirs, and
a pair of electrodes, one in each of the reservoirs to each end of the thread.

In some embodiments, there are at least four reservoirs for each sequence. These may include, for instance, a sample transfer reservoir and an operation reservoir, in addition to the reservoirs at each end of the threads which are associated with the electrodes.

The first and last reservoirs in each sequence may be shared between the sequences. That is, the same positive and negative electrodes (and associated reservoirs) may be shared between the sequences. Thus, each thread for each sequence may start in a shared, single reservoir at one end, and terminate in a shared, single reservoir at the opposite ends of the threads. The central reservoir (or reservoirs if more than one) positioned along the thread between each of the end reservoirs are separate for each thread (sequence) in the array. Alternatively, each sequence may comprise separate reservoirs and electrodes at each end of each thread. (Such an arrangement is illustrated in FIG. 4.) The system may further comprise a controller for controlling the application of a voltage potential across the electrophoresis matrix (thread), and performing any other steps such as electrophoretic separation and analysis.

It is noted that the system may comprise a cartridge that provides each of the features (i) to (iv) indicated above for the system. Thus, the cartridge may comprise a series of reservoirs (e.g. three, four or more reservoirs), a pair of electrodes, a thread and reagent pods. In alternative arrangements, the cartridge may comprise a series of at least four reservoirs, a thread connecting the reservoirs in series and reagent pods containing reagents suitable for each reservoir. One or more of the reservoirs may further comprise a magnetic bead for stirring contents in the reservoir. A magnetic bead may be positioned in an operation reservoir, as one example. A magnetic bead may additionally, or alternatively, be positioned in the sample transfer reservoir, if required for stirring the contents of that reservoir. As described in further detail below, the reagents in the reagent pods may include the required electrolyte, lysis buffer or reagents for performing a chemical reaction or aiding analysis as required in the associated reservoir.

An associated invention that has been developed by the applicant, which can be used in combination with the sample-transfer system or independently of the sample-transfer system, is a system that allows for one or more operations to be performed on the charged substance taken from the sample.

In one example of the third aspect of the invention, there is provided an electrophoresis system for performing an operation on a charged substance, the system comprising:
a first electrolyte reservoir comprising a first electrode;
a second electrolyte reservoir comprising a second electrode;
a thread extending between the first and second electrolyte reservoirs;
a sample loading reservoir positioned along the thread between the first and second electrolyte reservoirs for loading sample onto the thread;
a controller for controlling the application of an electric field across the thread so as to effect movement of the charged substance along the thread from the sample loading reservoir towards the second electrolyte reservoir; and
a operation zone positioned along the thread between the sample loading reservoir and the second electrolyte reservoir, at which an operation can be performed involving the charged substance.

As noted above, the system may include just one of the reservoirs selected from the sample loading reservoir and the operation reservoir, but in embodiments described below both reservoirs are described. The description should be read in light of this.

The operation zone is preferably a zone of the thread that is in the region of an operation reservoir. The operation reservoir is a reservoir that receives a liquid, such as an electrolyte and/or reagent, in the vicinity of which an operation can be performed on the charged substance. Examples of “operations” being performed involving the charged substance include analysis of the charged substance, coupling of the charged substance to a marker, a chemical reaction or a transformation involving the charged substance, and so forth. The sample loading reservoir is free of any electrode, as is the operation reservoir (when present).

The system may comprise further electrode-free reservoirs. Such reservoirs may enable the performance of more than one operation along the flow-path of the charged substance along the thread.

The system, or components of the system, may be provided in a cartridge format. In one example where the system includes four (or more) reservoirs, the cartridge for use in an electrophoresis instrument comprises:
a first electrolyte reservoir comprising a first electrode;
a second electrolyte reservoir comprising a second electrode;
a thread extending between the first and second electrolyte reservoirs;
a sample loading reservoir positioned along the thread between the first and second electrolyte reservoirs for loading sample onto the thread; and
a operation reservoir positioned along the thread between the sample loading reservoir and the second electrolyte reservoir within which an operation can be performed involving the charged substance.

The cartridge may further comprise electrolyte for each of the first electrolyte reservoir and the second electrolyte reservoir. The cartridge may additionally comprise electrically conductive reagent for the sample loading reservoir and the operation reservoir.

In some embodiments, the system includes an array comprising multiple threads, at least one each of the first and second electrolyte reservoirs and first and second electrodes, and multiple sets of said sample loading reservoirs and operation reservoirs. If provided in cartridge form, each cartridge may be for a single set, or a single cartridge may contain multiple sets of the reservoirs, electrodes and threads.

An array of such components allows for multiple parallel runs to be performed to achieve multiplexed or high throughput analysis. These may be performed on either a single sample (or single sample collector/swab) or form multiple samples (sample collectors/swabs). Multiplexed analysis has been performed by splitting a single thread into multiple pathways, where each pathway was used to determine a specific marker to provide a more holistic sample analysis and minimise the false positive and negative results that are often obtained when a single marker is analysed. High throughput analysis has been performed by recruiting multiple threads, substantially in parallel, in which each thread was used to perform analysis on an individual swab, minimising the average sample analysis time in situations such as epidemics and pandemics.

As indicated further above, microfluidic textile analytical devices have been restricted to the use of electrode-coupled initial and terminating reservoirs. However, the above-described configuration of the third aspect of the present application has been developed that allows the use of multiple electrode-free reservoirs, facilitating multi-step analysis and minimising the risks of electrode fouling. In key embodiments of the third aspect, there are provided additional reservoirs have been arranged between the initial and terminating electrodes such that they do not break the electro-fluidic circuit while also allowing independent activities, such as sample introduction, concentration, modification, detection, selective uptake or release, etc. The developed system can facilitate the use of microfluidic textile analytical devices in performing complex analytical procedures, which are often required in real-life settings. Moreover, since microfluidic textile analytical devices use high voltages, the availability of electrode free reservoirs for sample manipulation would also promote the generation of safer microfluidic textile analytical devices by preventing user exposure to the live electrodes. The present system also enables the user to easily perform an operation on a charged substance taken from a sample.

According to the fourth aspect, there is provided a method for performing an operation on a charged substance. In some embodiments, the method comprises:
providing a first electrolyte reservoir comprising a first electrode, a second electrolyte reservoir comprising a second electrode and a thread that extends between the first and second electrolyte reservoirs;
loading a charged substance onto the thread in a sample loading reservoir positioned along the thread between the first and second electrolyte reservoirs; and
moving the charged substance along the thread by electrophoresis from the sample loading reservoir towards an operation zone positioned downstream of the sample loading reservoir in the flow direction of the charged substance by applying an electric filed across the thread between the first and second electrodes; and
performing an operation involving the charged substance in the operation zone.

The thread may extend through an operation reservoir in which the operation zone of the thread is positioned, and the operation may be performed in the operation reservoir. There may be more than one such operation reservoir (or operation zones) traversed by the thread, and allowing for different operations to be performed in each of said reservoirs (or in each of said zones). The movement of the charged substance along the thread involves the application of an electric field across the thread.

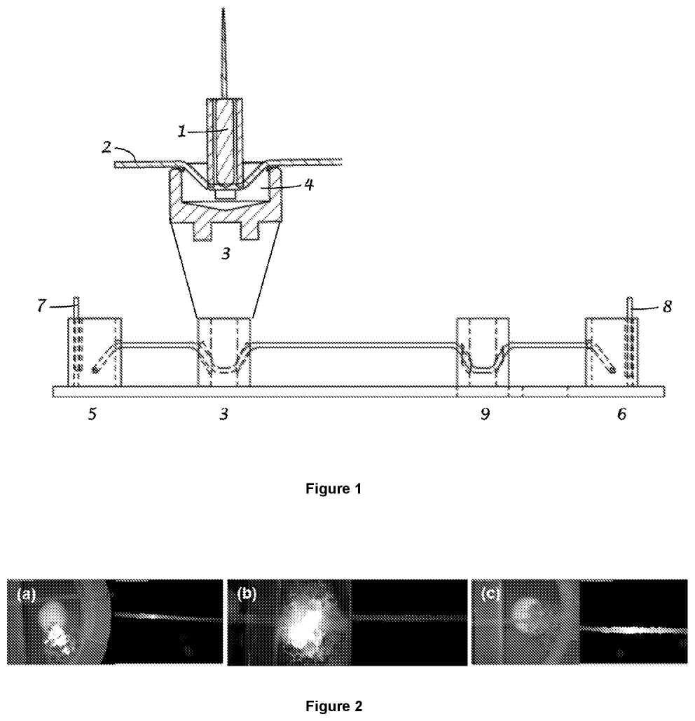

A suitable set-up for performing one or more of the above operations is shown in FIG. 1. In FIG. 1, components of the system are illustrated, including the following:
1. Swab
2. Thread
3. Sample transfer reservoir
4. Electrolyte
5. First electrolyte reservoir
6. Second electrolyte reservoir
7. First electrode
8. Second electrode
9. Operation reservoir

The system includes first and second electrolyte reservoirs (5, 6) each containing a first and second electrode, respectively. The first electrode is negatively charged, and the second electrode is positively charged through the application of an electric potential across the electrodes. The circuit is completed by the electrolytes that wet the thread (2). Any suitable electrolyte (or combination of electrolytes) can be used. In one example, the electrolytes include a terminating electrolyte which is in the first electrolyte reservoir (5) and the leading electrolyte which is in the second electrolyte reservoir (6). The sample transfer reservoir (3) also contains electrolyte, as does the operation reservoir (9). The thread is wetted by the electrolyte. The coating may be in any suitable state, such as liquid or gel, and therefore the thread may in an alternative example be coated by a conductive hydrogel coating.

A swab of a suitable material such as polyurethane or otherwise, is contacted with a surface to take a sample from the surface. The surface could be a part of a human such as the hand, mouth, tongue or otherwise, or the surface may be an inanimate surface. The swab (1) containing the sample (the sample comprising any number of components including one or more charged substances), is brought into direct contact with the thread (2) in the sample transfer reservoir (3). While in contact with the thread, an electric field (voltage potential) is applied across the electrodes (7, 8), for a time period to effect transfer of charged substances from the swab to the thread.

FIG. 2 shows a comparative test showing the impact that direct contact and applied electric field has on the transfer, compared to when the thread and swab are not contacted or the electric field is not applied. When the thread and swab are not contacted or the electric field is not applied the transfer occurs via the liquid medium (being an electrolyte) or through diffusion. In the top left image of FIG. 2 (a), a swab is shown following an attempted transfer of a fluorescent analyte, where the transfer took place by applying an electric filed across the thread, in the presence of an electrolyte, without direct contact between the swab and the thread. This resulted in only 6±3% of analyte transfer. The right-hand image of FIG. 2(a) shows the thread, where only a weak fluorescent band of the analyte was observed in line with its limited transfer. Similarly, FIG. 2(b) shows the swab (left side) and thread (right side) after an attempted transfer where the swab and the thread were in direct contact, however, an electric field was not applied across the thread. This resulted in only 5±1% of analyte transfer. In contrast, FIG. 2(c) shows the swab (left side) and thread (right side) following a transfer completed with direct contact between the swab and the thread while an electric field was applied across the thread. This time, a transfer of 94±1% was observed from the swab onto the thread.

Referring to FIG. 1, with the swab (1) still in contact with the thread (2), or following removal of the swab (1), the electric field is applied (or continues to be applied) to effect movement of the charged substance along the thread. Different charged substances (if more than one is present) move at different rates in view of their differing electroosmotic and electrophoretic mobilities. This effects separation of different charged substances (if multiple charged substances are present) and/or concentration of charged substances along the thread. The time period for a target charged substance, or analyte, to pass along the thread to the operation reservoir will be known for known substances, and thus the charged substance can be controlled to be positioned in the operation reservoir at a known time period for detection by a suitable detector mechanism, or for reaction, or otherwise.

By way of one example, the qualitative and quantitative analysis of the number and type of viral colonies present in the bodily fluids, such as cell lining, saliva, mucous, and blood can be performed using a combined approach of isotachophoresis (ITP) and reverse transcription loop-mediated isothermal amplification (RT-LAMP).

ITP facilitates the extraction and concentration of the nucleic acid content, and RT-LAMP facilitates selective amplification of the desired RNA for real-time quantitation.

Thread based ITP is used to perform sample clean-up, extraction, and pre-concentration of viral RNA. The user may position the collected swab into the second well, being the sample transfer reservoir (3) which may be viewed as a RNA extraction and lysis well, where the nucleic acid from the sample on the swab transfers directly to the thread (2) (otherwise referred to as a fibre). The absorbed RNA would then be focused into the third well, being the operation reservoir (9), using ITP.

RT-LAMP is an isothermal DNA amplification technique, which circumvents the need for cumbersome PCR instruments and is usually performed in simple Eppendorf vials by subjecting them to a constant temperature (usually 50-70° C.). Like PCR, the DNA amplification during RT-LAMP can be monitored using fluorescent tagging dyes, such as SYBR Green I (SG), which binds to DNA during its amplification and hence allows its easy quantification. Moreover, RT-LAMP technique only requires 15-20 minutes of analysis time as compared to more than 2 hours required for PCR, and the former also allows the use of multiple primers to provide high selectivity for the desired RNA strand.

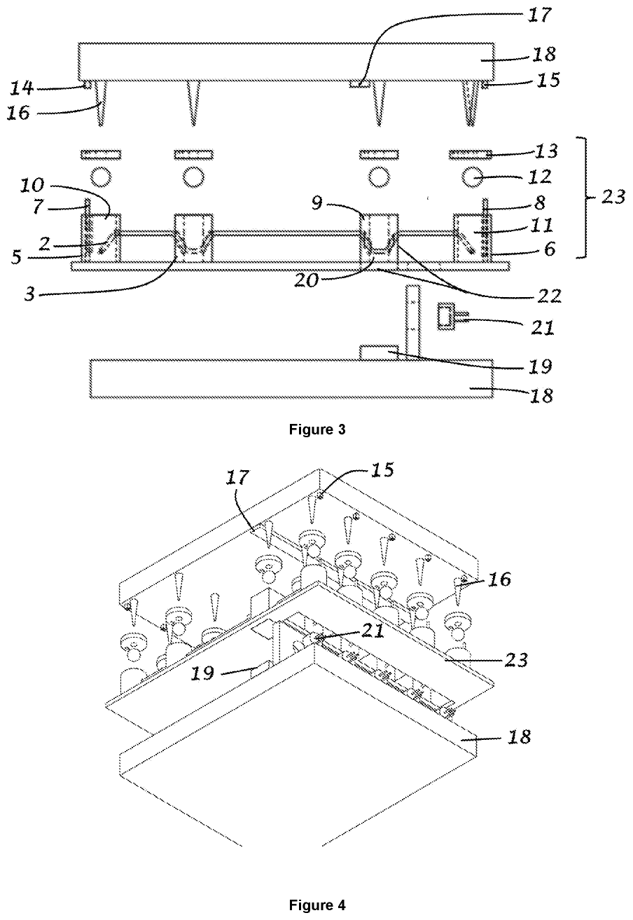

In one example, a cartridge-type system may be used. An example of one cartridge-type system is shown in FIG. 3. In FIG. 3, the same numerals are used to denote the same features as in FIG. 1, with the additional reference numerals indicating as follows:
10. Terminating electrolyte
11. Leading electrolyte
12. Reagent pods
13. Caps
14. First electrode socket
15. Second electrode socket
16. Retractable needles
17. Heater
18. Electronic chamber
19. Light source
20. RT-LAMP cuvette
21. Photodiode
22. Transparent windows

Each sample analysis cartridge consists of four wells (5, 3, 9, 6), two electrodes (7, 8), a thread (2), and four reagent pods (12). The cartridge body may be formed from plastic, with a thread, and metal electrodes (e.g. stainless steel) so that new cartridge can be used for each analysis to prevent any cross-contamination and hence false-positive results.

By positioning the electrodes in separate wells (5, 6), ITP can be used to focus RNA or other analytes on the thread.

Magnetic beads (not shown) may be pre-packaged into the second well (3) to allow stirring with a rotating magnet in the bottom panel of the electronic chamber (18), to assist with quick desorption and lysing. It is anticipated that magnetic beads are not specifically required in this well, but this is nevertheless an option. RNA would be focused in the third well, being the operation reservoir (9), where RT-LAMP would be performed using the overhead heater (17). The high speed of RT-LAMP would be improved due to pre-concentration of the RNA and its presence on a high surface-area to volume ratio thread. The DNA would bind with the available SYBR Green I (SG) fluorescent dye, and its quantity analysed using the lower light source (19) and individual photodiodes.

Precise quantities of the required reagents/electrolytes can be delivered in pre-packaged pods (12) within their respective wells. The pods may be pierced using retractable needles (16) to release the contents prior to the analysis.

FIG. 4 illustrates an array containing similar elements to those shown in FIG. 3. This arrangement contains an electronic chamber (18) which forms a part of the fixed instrument part of the system. There is a disposable cartridge (23) which contains an array of four wells and threads extending between the sets of four wells. The cartridge is designed for plug-and-play assembly with the fixed instrument.

Multiplexing enables analysis of multiple samples within the same total analysis time of 15-20 minutes. This enables mass testing especially at screening points such as airports, where samples can be collected as soon as passengers depart from the plane and their results would be available by the time they go through the immigration process.

Multiplexing can also be used to perform multi-stage analysis to identify all the positive cases, where one row could be used to screen for all SARS virus, the second row could be used to identify RdRP gene, and the third row could be used to perform a discriminatory test, as recommended.

### EXAMPLES

The inventions described herein will now be described in further detail with reference to the accompanying non-limiting examples, which demonstrate the efficacy of the invention.

### Example 1—Transfer of a Variety of Charged Substances from Swab to Thread Using Electrophoresis

Tests were conducted to show the efficacy of transfer of a variety of charged organic molecules of varying sizes from a swab to the nylon thread.

### Reagents and Materials

Tris-(hydroxyl methyl) amino-methane (TRIS), 4-(2-hydroxyethyl)-1-piperazineethanesulfonic acid (HEPES), hydrochloric acid, fluorescein sodium salt, coptisine chloride, palmatine chloride, and myoglobin, were obtained from Sigma-Aldrich (New South Wales, Australia). Berberine chloride, European Pharmacopoeia Reference Standard was purchased from EQDM Council of Europe (France). Chromeo 488 NHS-Ester was purchased from Santa Cruz Biotechnology (Santa Cruz, CA, USA). OraSwab plain was purchased from Confident Care products (New South Wales, Australia). Solutions were prepared in water from a Milli-Q Water Plus system from Millipore (Bedford, MA, USA), with a resistivity of 18.2 MΩ cm.

100% nylon (diameter (Ø) 803±53 μm, woolly nylon stretches overlocking thread, QA Thread, China) was used for the thread, otherwise referred to as the fibre-based microfluidic. Later examples involved the use of alternative thread materials. Threads (“fibres”) were washed in Milli-Q water and sonicated for 10 minutes 3 times to remove impurities on the surface. Subsequently, fibres were plasma treated by a vacuum plasma reactor (K1050X plasma asher (Quorum Emitech, UK)) to increase the wicking property, facilitating the sample application.

### Device Fabrication

Buffer reservoirs and a Lego type platform were designed with Fusion360 CAD software (Autodesk) and printed using an Eden 260VS (Stratasys, MN, USA) with the VeroClear build material, and SUP707 water-soluble support. The support material was cleaned with water and 2% NaOH as required. Subsequently, the reservoirs were rinsed and soaked in Milli-Q water for a day. The reservoirs were reused multiple times following a wash with water and 2% NaOH. Each reservoir consisted of a bridge to guide and submerge the thread in the buffer and two legs to mount them in the platform. The first and second reservoirs also consisted of a slot to hold the required electrode and a Lego type lock to adjust the thread's position and tension.

The assembly procedure involved two steps. Firstly, buffer reservoirs were inserted into the base according to the desired format and length. Secondly, the thread (fibre) was tensioned between the buffer reservoirs. In all experiments, fibres were kept wet with leading electrolyte solution during the electrophoresis process. The direction of electrophoretic migration was dictated by the analyte's charged state and the polarity of the electrodes in the first and second reservoirs.

Fluorescence images were obtained using a USB microscope AM4113T-GFBW (Dino-Lite Premier, Clarkson, WA, Australia) equipped with a blue light-emitting diode for excitation and a 510 nm emission filter. The microscope was controlled using DinoCapture 2.0 software. Fluorescence intensities of images and videos were processed with Image J software for the quantification of target fluorescent analytes.

A high voltage power supply was used for the introduction of voltage for all the fibre-based ITP experiments. For negatively charged analytes, such as fluorescein and myoglobin, the experiments were carried out in anodic mode (cathode in the inlet and anode in the outlet buffer reservoirs), and vice-versa for the positively charged analytes, such as alkaloids. The system was controlled using a 12-Bit, 10 KS/s multifunction DAQ system (USB-6008 OEM, National Instruments, Austin, TX, USA).

### Fibre-Based Isotachophoresis (ITP) Operation

In the case of fluorescein and myoglobin, a 5 mM TRIS/2.5 mM HEPES solution was used as the terminating electrolyte (TE), and 20 mM TRIS/10 mM HCl solution was used as the leading electrolyte (LE). While in the case of alkaloids, a 20 mM β-alanine solution was used as TE and 20 mM potassium acetate solution was used as the LE. The electroosmotic flow was suppressed by adding 0.1% PVP to the TE and LE solutions. After assembling the fibre-based ITP setup and placing the electrodes, 500 μL of TE and LE were added to the first and second reservoirs, respectively. The fibres were then wetted with the LE. The samples were swabbed form their 2 μL droplets of the desired concentration, which were previously spread over a glass slide. Before applying the voltage, the system was equilibrated for 1 minute to reduce the capillary action along the fibre. Finally, constant voltage or current was applied to initiate the ITP procedures.

### Results

Molecules that were transferred from the swab to the nylon thread through direct contact of the swab to the nylon thread, in the presence of an electrolyte and through the application of a voltage potential across the thread included fluorescein, alkaloids (in particular, a mixture of coptisine, palmatine, berberine) and protein (chromeo 488 NHS ester-labelled myoglobin). The results are shown in FIG. 5. In FIG. 5(a), the left-hand image shows a fluorescent image of the swab after the transfer is effected by electrophoresis, and the right-hand image (thread line) shows the positioning of the fluorescent fluorescein on the thread post-transfer and post-electrophoretic focusing. FIG. 5(b) shows the swab for the alkaloid composition post-transfer (left-half image) and the right-half image shows the alkaloids on the thread post-transfer and post-focusing. FIG. 5(c) shows the swab for the labelled protein post-transfer on the left, and the thread post-transfer on the right showing the presence of the protein. A sample volume of 2 μL was swabbed; the concentration of the analytes ranged from 10-100 ppm.

The molecular weights of these analytes range from 300 g/mol to 17000 g/mol. An instantaneous transfer was observed for all three analytes, and they were further focused on the thread (in a concentrated band) using isotachophoresis within 2-3 minutes.

An estimate of the percentage transfer was made based on the fluorescence in microscopic images. The images suggest that when the swab is in direct contact with the thread, this results in 30 and 40 times higher recovery for fluorescein and alkaloids, respectively, as compared to when the swab was present isolated in the same buffer reservoir (indirect contact). No observable transfer of myoglobin was observed during indirect contact. In the case of direct contact, a transfer of more than 90% was observed for all three analytes.

### Example 2—Transfer of a Charged Substance from a Swab to Different Thread Materials Using Electrophoresis

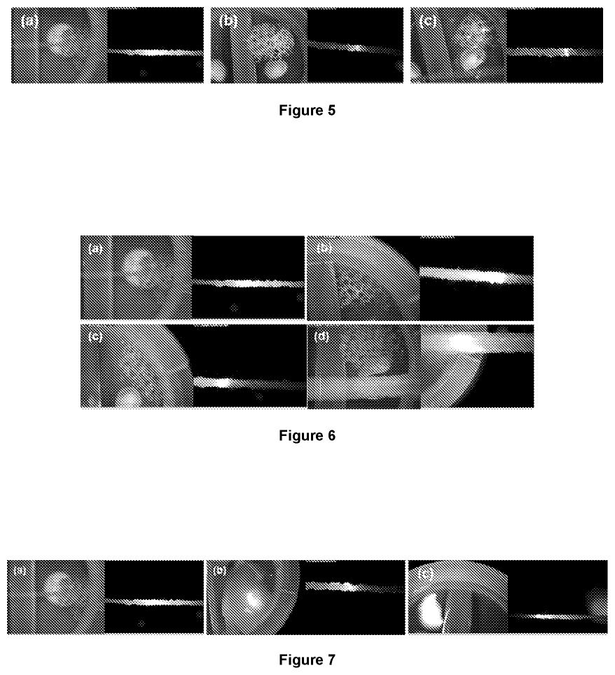

Tests were conducted to show the efficacy of transfer of fluorescein, as an exemplary charged substance, to threads of different materials, including nylon, mercerised cotton, cotton and polyester. The results are shown in FIG. 6—(a) is nylon, (b) is mercerised cotton, (c) is cotton (not mercerised) and (d) is polyester. In each sub-figure, a fluorescent image of the swab after the transfer has been shown on the left and a fluorescent image of the transferred and focused analyte band on the thread is shown on the right. A sample volume of 2 μL was swabbed; the concentration of fluorescein was 10 ppm. Hydrophilic yarns, such as mercerised cotton and cotton, do not require any pre-treatment before their use, whereas, hydrophobic threads, such as nylon and polyester, benefit from pre-treatment to impart wettability to the thread. Hence, untreated mercerised cotton and cotton were used. Nylon was treated with air plasma. The treatment time was 90 seconds. Un-treated hydrophobic thread, polyester (as shown in FIG. 6(d)) resulted in lower analyte mobility, however, still complete transfer of the analyte was observed. Quantitative analysis of the transfer suggested 94±1%, 97±2%, 98±1%, and 80±7% transfer onto nylon, mercerised cotton, cotton, and polyester, respectively.

### Example 3—Transfer of a Charged Substance from Different Swab Materials onto a Thread Using Electrophoresis

Tests were conducted to show the efficacy of transfer of fluorescein, as an exemplary charged substance, from different swab materials, onto nylon thread (as an exemplary thread material). The three tested swab materials were polyurethane, cotton, and flocked nylon. The results are shown in FIG. 7 where (a) is polyurethane, (b) is cotton, and (c) is flocked nylon. In each sub-figure, a fluorescent image of the swab after the transfer has been shown on the left and a fluorescent image of the transferred and focused analyte band on the thread is shown on the right. A sample volume of 2 μL was swabbed; the concentration of fluorescein was 10 ppm. A transfer of 94±1% was observed from the polyurethane swab. A transfer amount of 47±7% of fluorescein was observed from the cotton swab. A transfer of 97±2% was observed from the flocked nylon swab. The high fluorescence observed in the flocked nylon swab after transfer was due to the high background fluorescence of the swab itself and not the remaining sample. The limited transfer from the cotton swab is presumably due to an interaction between fluorescein with cotton.

### Example 4—Modified Sample Transfer Reservoir Design

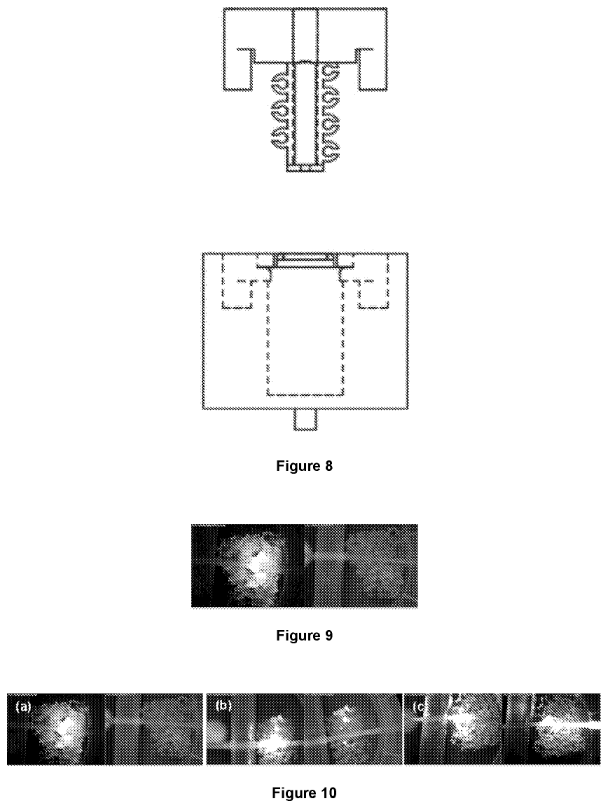

In FIG. 1, one version of the sample transfer reservoir is shown. In FIG. 8, an alternative design is illustrated. In the arrangement shown in FIG. 8, there is shown a lower section and an upper section. The lower section includes the reservoir opening, with two bores for receiving two corresponding pins located on the upper section. The upper section includes a central plunger with a central slot and seven clips for receiving a thread. The swab may be inserted into the plunger, and the thread wrapped in a spiral fashion through the clips and around the swab. The design allows for greater contact between the thread and the swab, which aids in the transfer of charged substance from the swab to the thread, and for the swab to be moved independently of the reservoir, allowing greater operational freedom.

### Example 5—Transfer of a Charged Substance in the Absence of any Additional Liquid in the Sample Transfer Reservoir

Tests were conducted to show the efficacy of transfer of fluorescein (as an exemplary charged substance) from polyurethane swab (as an exemplary swab material) onto nylon thread (as an exemplary thread material) in the absence of any liquid (buffer, electrolyte or otherwise) in the sample transfer reservoir while the swab was still in direct contact with the thread. While there was no bulk liquid (electrolyte, buffer or otherwise) present, the nylon thread was wetted by leading electrolyte (as described in the “Reagents and Materials” section above), providing the electroresis matrix (thread) with the required presence of electrolyte. The results are shown in FIG. 9 where a fluorescent image of the swab and thread junction before starting the transfer is shown on the left, and a fluorescent image of the swab and thread junction at the end of the transfer experiment is shown on the right. A sample volume of 2 μL was swabbed; the concentration of fluorescein was 10 ppm. A transfer of 97±2% was observed, which is similar to the transfer observed in the presence of a buffer in the sample transfer reservoir. This further confirms the direct transfer of charged analytes from a swab onto an electrophoretic matrix (in the presence of electrolyte) and simplifies the system by omitting the need for any additional medium (such as bulk liquid electrolyte) in the sample transfer zone.

### Example 6—Transfer of a Charged Substance from Different Sample and Swab States in the Absence of any Additional Liquid in the Sample Transfer Reservoir

Tests were conducted to show the efficacy of transfer of fluorescein (as an exemplary charged substance) from polyurethane swab (as an exemplary swab material) onto nylon thread (as an exemplary thread material) in the absence of any liquid in the sample transfer reservoir. Different combinations of sample and swab states were studied, i.e. (a) liquid sample swabbed with a swab pre-wetted with the terminating electrolyte (the default state), (b) liquid sample swabbed with a dry swab, and (c) powder sample swabbed with a dry swab. The results are shown in FIG. 10, where (a) is a liquid sample with a pre-wetted swab, (b) is a liquid sample with a dry swab, and (c) is a powder sample with a dry swab. In each combination for this example, the nylon thread was wetted by leading electrolyte (electrolyte as described in the “Reagents and Materials” section above), providing the electroresis matrix (thread) with the required electrolyte to support the transfer, and the electric field was applied across the electrophoresis matrix to effect the transfer. In each sub-figure, a fluorescent image of the swab and thread junction before starting the transfer is shown on the left and a fluorescent image of the swab and thread junction at the end of the transfer experiment is shown on the right. In the case of the liquid samples, a sample volume of 2 μL was swabbed with a fluorescein concentration of 10 ppm. In the case of the powder samples, 1 mg sample was swabbed. A transfer of 97±2% was observed when a liquid sample on a pre-wetted swab was in direct contact with the thread. A transfer of 93±1% was observed when a liquid sample on a dry swab was in direct contact with the thread. A significant transfer was observed even when a powder sample on a dry swab was in direct contact with the thread. The transfer in the case of the powder sample swabbed with a dry swab in the absence of any bulk liquid medium (but in the presence of liquid electrolyte wetting the thread only) suggests high robustness and wide applicability of the system since it minimises any pre-conditioning of either the sample, swab, or the sample transfer zone. The transfer is expected to occur (without being limited by the theoretical considerations) due to the solvation of the analyte molecules as they encounter the liquid medium (electrolyte) on the thread. The solvated molecules are then electrokinetically transferred from the swab onto the thread.

### Example 7—Transfer of a Charged Substance when Dried Liquid Samples are Swabbed from Different Materials with Different Swab States

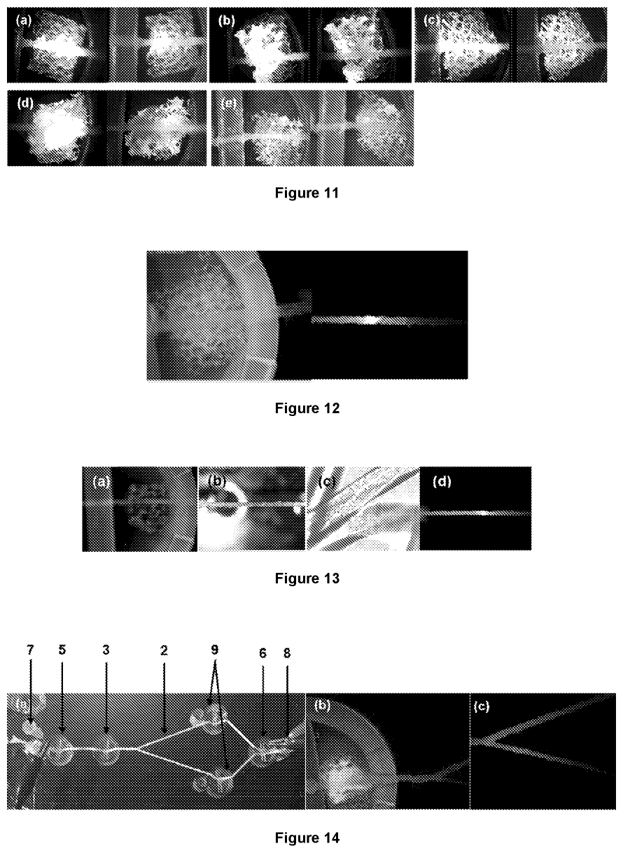

Tests were conducted to show the efficacy of transfer of fluorescein (as an exemplary charged substance) when its dried liquid sample was swabbed with a polyurethane swab (as an exemplary swab) from the surfaces of different types of material, onto a nylon thread (as an exemplary thread). Three different materials, i.e. (a) plastic, (b) metal, and (c) wood, were swabbed with two different hydrated states of the swab, i.e. (a) pre-wet state and (b) dry state. The results are shown in FIG. 11, where (a) is a plastic surface swabbed with a pre-wetted swab, (b) is a plastic surface swabbed with a dry swab, (c) is a metallic surface swabbed with a pre-wetted swab, (d) is a metallic surface swabbed with a dry swab, and (e) is a wooden surface swabbed with a pre-wetted swab. In each sub-figure, a fluorescent image of the swab and thread junction before starting the transfer is shown on the left and a fluorescent image of the swab and thread junction at the end of the transfer experiment is shown on the right. A sample volume of 10 μL with a fluorescein concentration of 100 ppm was dried on each material. A transfer of more than 90% was observed in each case based on the fluorescent microscope images. In most cases, a pre-wet swab resulted in better sample collection, primarily due to the collected sample's dried nature. However, no significant difference was observed in the transfer of the collected charged analyte from the dry or pre-wet states of the swabs.

### Example 8—Transfer of a Charged Substance in the Presence of a Complex Sample Matrix

Tests were conducted to show the efficacy of transfer of fluorescein (as an exemplary charged substance) in the presence of different sample matrices, from a polyurethane swab (as an exemplary swab), onto a nylon thread (as an exemplary thread). The results are shown in FIG. 12, where a fluorescent image of the swab and thread junction before starting the transfer from spiked saliva samples in the presence of a cell lysis buffer is shown on the left and a fluorescent image of the swab and thread junction at the end of the transfer experiment is shown on the right. A sample volume of 2 μL was swabbed; the concentration of fluorescein was 10 ppm. A transfer of 95±2% was observed in the presence of the cell lysis buffer. A transfer of 92±3% was observed in the presence of saliva and the cell lysis buffer. The percentage transfer did not show any significant difference in the presence or absence of a complex sample matrix. However, the rate of transfer was lower in the presence of a sample matrix compared to the absence of any sample matrix.

### Example 9—Transfer, Separation, and Concentration of a Charged Substance from a Complex Sample

Tests were conducted to show the efficacy of transfer, separation, and concentration of fluorescently tagged DNA (as an exemplary charged substance) from defibrillated sheep blood (as an exemplary complex sample) using a polyurethane swab (as an exemplary swab) onto a nylon thread (as an exemplary thread). The results are shown in FIG. 13, where (a) is the image of the swabbed spiked blood sample, (b) is the image of the separated blood cells and haemoglobin on the thread, (c) is the microscopic image of the lysed cells on the thread, and (d) is the fluorescent image of the focused fluorescently tagged DNA band on the thread. The blood sample was spiked with 20% v/v 1 nm fluorescently tagged DNA, and a sample volume of 10 μL was swabbed from a glass slide. The sample transfer reservoir was filled with the background electrolyte and a cell lysis buffer. The fluorescently tagged DNA was successfully transferred from the swabbed sample, separated from the other blood components, and focused into a thin band on the thread. Also, the blood cells were successfully lysed using the cell lysis buffer, confirming the ability to modify the swabbed sample simultaneously during the transfer.

### Example 10—Splitting the Transferred Substance into Multiple Threads Downstream for Multiplexed Analysis

Tests were conducted to show the efficacy of transfer and splitting of fluorescein (as an exemplary charged substance) sample from a single polyurethane swab (as an exemplary swab material) onto two nylon threads (as an exemplary thread material). The results are shown in FIG. 14, where (a) is the arrangement of the splitting threads with their individual operational reservoirs, (b) is the image of the swab in direct contact with a single thread, and (c) is the fluorescent image of the analyte split onto two threads. A sample volume of 2 μL was swabbed; the concentration of fluorescein was 10 ppm. The fluorescent analyte was found to split between two different threads as 50±3% on each. This allows multiple independent analyses along different threads on the same sample.

Various modifications may be made to the embodiments described above with reference to the Figures and Examples without departing from the spirit and scope of the invention. In the present specification and claims, the term “comprising” or variations such as “comprises” or “comprising” is used in an inclusive sense, i.e. to specify the presence of the stated features but not to preclude the presence or addition of further features.

### Items:

1. A method for the transfer of a charged substance from a sample on a sample collector to an electrophoresis matrix, the method comprising:
   contacting the sample collector containing a sample including the charged substance with the electrophoresis matrix in the presence of an electrolyte;
   applying an electric field across the electrophoresis matrix to effect transfer of the charged substance from the sample collector to the electrophoresis matrix.
2. The method of item 1, wherein the electrophoretic matrix comprises a thread.
3. The method of item 1 or item 2, wherein the sample collector is a swab.
4. The method of any one of the preceding items, wherein at least 50% of the charged substance is transferred from the sample collector to the electrophoresis matrix on the application of an electric field.
5. The method of item 4, wherein at least 60%, or at least 70%, or at least 80% or at least 90% of the charged substance is transferred from the sample collector to the electrophoresis matrix on the application of an electric field.
6. The method of any one of the preceding items, further comprising moving the charged substance along the electrophoresis matrix through electrophoresis or isotachophoresis away from the location of initial transfer of the charged substance onto the electrophoresis matrix.
7. The method of item 6, wherein the charged substance is moved along the electrophoresis matrix to an operation reservoir, in which an operation is performed on the charged substance.
8. The method of any one of the preceding items, further comprising performing an operation on the charged substance after its transfer to the electrophoresis matrix, the operation selected from the group consisting of chemical analysis, detection, coupling of the charged substance to another chemical or chemical modification of the charged substance.
9. The method of item 6 or item 7, further comprising performing an operation on the charged substance selected from the group consisting of chemical analysis, detection, coupling of the charged substance to another chemical or chemical modification of the charged substance, following the movement of the charged substance along the electrophoresis matrix by electrophoresis or isotachophoresis.
10. The method of item 6, further comprising:
subjecting the charged substance transferred onto the electrophoresis matrix to electrophoresis to separate a target analyte that forms a component of the charged substance into a focused band on the electrophoresis matrix.
11. The method of item 10, further comprising detecting for the presence of the target analyte.
12. The method of item 11, wherein the detecting step comprises detecting for the target analyte as the focused band of the target analyte on the electrophoresis matrix passes through a detection zone of the electrophoretic matrix.
13. The method of any one of the preceding items, wherein the electrophoresis matrix is open to the environment to enable modifications to be made on the electrophoresis matrix.
14. The method of item 13, wherein the electrophoresis matrix is in the form of a thread, and the thread is cut to isolate a portion of the thread containing the charged substance.
15. The method of item 14, further comprising recovery of the charged substance from the cut portion of the thread.
16. The method of any one of the preceding items, wherein the electric field is applied across the electrophoresis matrix via a pair of electrodes positioned to apply the electric field along the electrophoresis matrix.
17. The method of item 16, wherein a first electrode of the pair of electrodes is positioned in a first electrolyte reservoir, and the second electrode of the pair of electrodes is positioned in a second electrolyte reservoir, and wherein the electrophoresis matrix extends between the first and second electrolyte reservoirs and is in electrical connection with the first and second electrodes.
18. The method of item 17, comprising transfer of the charged substance from the sample collector to the electrophoresis matrix either (i) in said first reservoir or (ii) in a first location along the electrophoresis matrix between a first electrode and a second electrode.
19. The method of item 18, further comprising moving the charged substance along the electrophoresis matrix from the first reservoir or the first location along the electrophoresis matrix to an operations reservoir for the performance of an operation on the charged substance.
20. A system for the transfer of a charged substance from a sample on a sample collector to an electrophoresis matrix, the system comprising components including:
   a sample transfer reservoir;
   an electrophoresis matrix comprising a thread positioned through the sample transfer reservoir;
   a pair of electrodes positioned to enable the application of an electric field across the thread;
wherein the components are positioned such that the sample collector contacts the thread during the application of an electric field across the thread to enable transfer of the charged substance from the sample collector to the thread.
21. The system of item 20, comprising a sample transfer receiver for the receipt of the sample collector to facilitate contact of the sample collector with the thread during application of the electric field across the thread.
22. The system of item 20 or item 21, comprising a first electrolyte reservoir in which a first of the pair of electrodes is positioned, and a second electrolyte reservoir in which a second of the pair of electrodes is positioned, with the thread extending between the first and second electrolyte reservoirs, wherein the sample transfer reservoir is positioned between the first and second electrolyte reservoirs along the thread.
23. The system of any one of items 20 to 22, further comprising an operation reservoir positioned along the thread between the sample transfer reservoir and second electrolyte reservoir at which the operation involving the charged substance can be performed following movement of the charged substance to the operation reservoir along the thread.
24. The system of any one of items 20 to 23, in the form of an array comprising multiple sets of said reservoirs and threads, and at least one of said pairs of electrodes.
25. An electrophoresis system comprising:
   a first electrolyte reservoir comprising a first electrode;
   a second electrolyte reservoir comprising a second electrode;
   a thread extending between the first and second electrolyte reservoirs;
   a third reservoir through which the thread passes; and
   a controller for controlling the application of an electric field across the thread so as to effect movement of a charged substance along the thread;
   wherein the third reservoir enables either (a) the charged substance to be loaded onto the thread in the third reservoir, or (b) an operation to be performed on the charged substance transferred to the third reservoir following movement of the charged substance along the thread on the application of the electric field.
26. The system of item 25, wherein the third reservoir is positioned between the first and second electrolyte reservoirs.
27. The system of item 25 or item 26, wherein the movement of the charged substance along the thread is in a direction towards the second electrolyte reservoir.
28. The system of any one of items 25 to 27, wherein the third reservoir is a sample loading reservoir in which sample can be loaded onto the thread on the application of an electric field, and the system further comprises an operation reservoir positioned along the thread between the sample loading reservoir and second electrolyte reservoir at which the operation involving the charged substance can be performed.
29. A cartridge for use in an electrophoresis instrument, the cartridge comprising:
   a first electrolyte reservoir comprising a first electrode;
   a second electrolyte reservoir comprising a second electrode;
   a thread extending between the first and second electrolyte reservoirs; and
   a third reservoir through which the thread passes between the first and second electrolyte reservoirs, within which either (a) a charged substance may be loaded onto the thread, or (b) an operation can be performed on charged substance following transfer of the charged substance along the thread to the third reservoir.
30. The cartridge of item 29, wherein the third reservoir is a sample loading reservoir, and the cartridge further comprises an operation reservoir positioned between the sample loading reservoir and the second electrolyte reservoir within which an operation can be performed on the charged substance following transfer from the sample loading reservoir to the operation reservoir.
31. The cartridge of item 30, the cartridge including an array comprising multiple sets of threads, multiple sets of said reservoirs, and at least one each of said first and second electrolyte reservoirs and said first and second electrodes.
32. A method for performing an operation on a charged substance, the method comprising:
   providing a first electrolyte reservoir comprising a first electrode, a second electrolyte reservoir comprising a second electrode and a thread that extends between the first and second electrolyte reservoirs;
   loading a charged substance onto the thread;
   moving the charged substance along the thread to an operation reservoir positioned downstream in the flow direction of the charged substance, by applying an electric field across the thread between the first and second electrodes; and
   performing an operation involving the charged substance in the operation reservoir.
33. The method of item 32, wherein the loading of the charged substance onto the thread comprises contacting a sample collector containing a sample including the charged substance with the thread in the presence of an electrolyte, and applying an electric field across the thread to effect transfer of the charged substance from the sample collector to the thread.
34. The method of item 33, wherein the sample collector is a swab.
35. The method of any one of items 33 to 34, wherein at least 50%, or at least 60%, or at least 70%, or at least 80% or at least 90% of the charged substance is transferred from the sample collector to the electrophoresis matrix on the application of an electric field.
36. The method of any one of items 32 to 35, wherein the operation is selected from the group consisting of chemical analysis, detection, coupling of the charged substance to another chemical or chemical modification of the charged substance.
37. The method of any one of items 32 to 36, wherein the step of moving the charged substance along the thread comprises separating a target analyte that forms a component of the charged substance into a focused band on the thread, and the step of performing an operation involving the charged substance comprises detecting for the presence of the target analyte.
38. The method of any one of clams 32 to 36, wherein the thread is open to the environment to enable modifications to be made on the thread.
39. The method of item 38, wherein thread is cut to isolate a portion of the thread containing the charged substance.
40. The method of item 39, further comprising recovery of the charged substance from the cut portion of the thread.
41. The method of any one of items 32 to 40, wherein the loading of the charged substance onto the thread takes place in said first electrolyte reservoir.
42. The method of any one of items 32 to 40, wherein the loading of the charged substance onto the thread takes place in a sample loading reservoir that is separate to the first electrolyte reservoir.

1. A method for transfer of a charged substance from a sample on a sample collector to an electrophoresis matrix, the method comprising:
contacting the sample collector containing a sample including the charged substance with the electrophoresis matrix in the presence of an electrolyte;
applying an electric field across the electrophoresis matrix to effect transfer of the charged substance from the sample collector to the electrophoresis matrix.

2. The method of claim 1, wherein the electrophoretic matrix comprises a thread.

3. The method of claim 1, wherein the sample collector is a swab.

4. The method of claim 1, wherein at least 50% of the charged substance is transferred from the sample collector to the electrophoresis matrix on the application of an electric field.

5. (canceled)

6. The method of claim 1, further comprising moving the charged substance along the electrophoresis matrix through electrophoresis or isotachophoresis away from the location of initial transfer of the charged substance onto the electrophoresis matrix.

7. The method of claim 6, wherein the charged substance is moved along the electrophoresis matrix to an operation reservoir, in which an operation is performed on the charged substance, wherein the operation is selected from the group consisting of chemical analysis, detection, coupling of the charged substance to another chemical or chemical modification of the charged substance.

8. The method of claim 1, further comprising performing an operation on the charged substance after its transfer to the electrophoresis matrix, the operation selected from the group consisting of chemical analysis, detection, coupling of the charged substance to another chemical or chemical modification of the charged substance.

9. (canceled)

10. The method of claim 6, further comprising:
subjecting the charged substance transferred onto the electrophoresis matrix to electrophoresis to separate a target analyte that forms a component of the charged substance into a focused band on the electrophoresis matrix.

11. The method of claim 10, further comprising detecting for the presence of the target analyte, wherein the detecting step comprises detecting for the target analyte as the focused band of the target analyte on the electrophoresis matrix passes through a detection zone of the electrophoretic matrix.

12. (canceled)

13. The method of claim 1, wherein the electrophoresis matrix is open to the environment to enable modifications to be made on the electrophoresis matrix.

14. The method of claim 13, wherein the electrophoresis matrix is in the form of a thread, and the thread is cut to isolate a portion of the thread containing the charged substance.

15. The method of claim 14, further comprising recovery of the charged substance from the cut portion of the thread.

16. The method of claim 1, wherein the electric field is applied across the electrophoresis matrix via a pair of electrodes positioned to apply the electric field along the electrophoresis matrix, wherein a first electrode of the pair of electrodes is positioned in a first electrolyte reservoir, and the second electrode of the pair of electrodes is positioned in a second electrolyte reservoir, and wherein the electrophoresis matrix extends between the first and second electrolyte reservoirs and is in electrical connection with the first and second electrodes.

17. (canceled)

18. The method of claim 16, comprising transfer of the charged substance from the sample collector to the electrophoresis matrix either (i) in said first reservoir or (ii) in a first location along the electrophoresis matrix between a first electrode and a second electrode, and further comprising moving the charged substance along the electrophoresis matrix from the first reservoir or the first location along the electrophoresis matrix to an operations reservoir for the performance of an operation on the charged substance.

19. (canceled)

20. A system for the transfer of a charged substance from a sample on a sample collector to an electrophoresis matrix, the system comprising components including:
a sample transfer reservoir;
an electrophoresis matrix comprising a thread positioned through the sample transfer reservoir;
a pair of electrodes positioned to enable the application of an electric field across the thread;
wherein the components are positioned such that the sample collector contacts the thread during the application of an electric field across the thread to enable transfer of the charged substance from the sample collector to the thread.

21. The system of claim 20, comprising a sample transfer receiver for the receipt of the sample collector to facilitate contact of the sample collector with the thread during application of the electric field across the thread.

22. The system of claim 20, comprising a first electrolyte reservoir in which a first of the pair of electrodes is positioned, and a second electrolyte reservoir in which a second of the pair of electrodes is positioned, with the thread extending between the first and second electrolyte reservoirs, wherein the sample transfer reservoir is positioned between the first and second electrolyte reservoirs along the thread.

23.-28. (canceled)

29. A cartridge for use in an electrophoresis instrument, the cartridge comprising:
a first electrolyte reservoir comprising a first electrode;
a second electrolyte reservoir comprising a second electrode;
a thread extending between the first and second electrolyte reservoirs; and
a third reservoir through which the thread passes between the first and second electrolyte reservoirs, within which either (a) a charged substance may be loaded onto the thread, or (b) an operation can be performed on charged substance following transfer of the charged substance along the thread to the third reservoir.

30. The cartridge of claim 29, wherein the third reservoir is a sample loading reservoir, and the cartridge further comprises an operation reservoir positioned between the sample loading reservoir and the second electrolyte reservoir within which an operation can be performed on the charged substance following transfer from the sample loading reservoir to the operation reservoir.

31. The cartridge of claim 30, the cartridge including an array comprising multiple sets of threads, multiple sets of said reservoirs, and at least one each of said first and second electrolyte reservoirs and said first and second electrodes.

32.-42. (canceled)
